(19) United States
(12) Patent Application Publication
(10) Pub. No.: US 2009/0181935 A1
(43) Pub. Date: Jul. 16, 2009

(54) COMPOSITIONS COMPRISING AN ANTIMUSCARINIC AND A LONG-ACTING BETA-AGONIST

(75) Inventors: Gino Villetti, Parma (IT); Roberta Razzetti, Parma (IT)

Correspondence Address: OBLON, SPIVAK, MCCLELLAND MAIER & NEUSTADT, P.C., 1940 DUKE STREET, ALEXANDRIA, VA, 22314, US

(73) Assignee: Chiesi Farmaceutici S.p.A., Parma (IT)

(21) Appl. No.: 12/343,882

(22) Filed: Dec. 24, 2008

(30) Foreign Application Priority Data
Jan. 15, 2008 (EP) 08000601.8

(51) Int. Cl. A61K 31/439; A61K 31/58; A61K 31/56; A61P 11/00

(52) U.S. Cl. 514/171; 514/305

(57) ABSTRACT

Compositions which comprise a combination of a salt of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane, and a long-acting phenylalkylamino beta₂-agonist are effective for the prevention and treatment of inflammatory or obstructive airways diseases.

### CROSS REFERENCES TO RELATED APPLICATIONS

This application claims priority to European Patent Application No. 08000601.8, filed on Jan. 15, 2008, which is incorporated herein by reference in its entirety.

### BACKGROUND OF THE INVENTION

1. Field of the Invention

The present invention relates to compositions and kit thereof, and to their use in the prevention and/or treatment of an inflammatory or obstructive airways disease. The present invention also relates to methods for the prevention and/or treatment of an inflammatory or obstructive airways disease.

2. Discussion of the Background

Quaternary ammonium salts acting as muscarinic receptors antagonists are currently used in therapy to induce bronchodilation for the treatment of respiratory diseases, and in particular inflammatory or obstructive airway diseases such as asthma and chronic obstructive pulmonary disease (COPD).

For treating chronic diseases, it is often desirable to utilize antimuscarinic drugs with a long-lasting effect. This ensures that the concentration of the active substance necessary for achieving the therapeutic effect is present in the lungs for a long period of time, without the need for the active substance to be administered repeatedly and too frequently.

In particular, it would be desirable to utilize antimuscarinic drugs which are therapeutically efficacious upon administration by inhalation once a day. In order to fulfill such a requirement, antimuscarinic drugs shall exhibit good selectivity for M3 muscarinic receptors, and slow dissociation from them.

Recently, it has been reported that tiotropium bromide, the first drug in a new generation of antimuscarinic drugs, exhibits a very slow dissociation from M3 receptors, behaviour thought to account for its long lasting activity. However tiotropium bromide still retains slow dissociation kinetics for the M2 muscarinic receptors. Since M2 receptors are a major population in the cardiac muscle, a therapy with said drug might be accompanied by undesired cardiac side effects.

The quaternary ammonium salt of 3-[[[(3-fluorophenyl)[(3,4,5-trifluoro phenyl)methyl]amino]carbonyl]oxy]-1-[2-oxo-2-(2-thienyl)ethyl]-1-azoniabicyclo[2.2.2]octane (hereinafter referred to as active compound 1) is a novel compound which has been disclosed in the co-pending Patent Application no. PCT/EP2007/057585, which is incorporated herein by reference in its entirety. The active compound 1 has the following chemical structure:

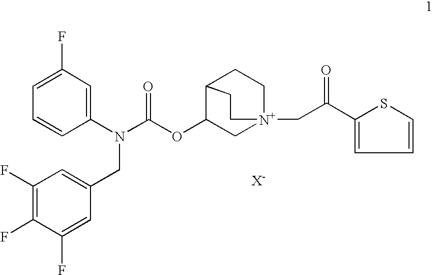

wherein X⁻ is a pharmaceutically acceptable anion, preferably selected from the group consisting of chloride, bromide, iodide, sulfate, phosphate, methanesulfonate, nitrate, maleate, acetate, citrate, fumarate, tartrate, oxalate, succinate, benzoate, and p-toluenesulfonate.

### SUMMARY OF THE INVENTION

Accordingly, it is one object of the present invention to provide novel compositions which are useful for the prevention and/or treatment of an inflammatory or obstructive airways disease.

It is another object of the present invention to provide novel kits which are useful for the prevention and/or treatment of an inflammatory or obstructive airways disease.

It is another object of the present invention to provide novel methods for the prevention and/or treatment of an inflammatory or obstructive airways disease.

These and other objects, which will become apparent during the following detailed description, have been achieved by the inventors' discovery that compositions which comprise a pharmaceutically acceptable salt of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane (active compound 1) in combination with a long-acting phenylalkylamino beta₂-agonist (active compound 2) or a pharmaceutically acceptable salt thereof are particularly useful for the prevention and/or treatment of an inflammatory or obstructive airways disease.

Thus, in a first embodiment, the present invention provides compositions which comprise a pharmaceutically acceptable salt of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane (active compound 1) in combination with a long-acting phenylalkylamino beta₂-agonist (active compound 2) or a pharmaceutically acceptable salt thereof.

In a preferred embodiment of the present invention, the composition comprises the active compound 1 in combination with formoterol or carmoterol as compound 2.

In the compositions mentioned above, the active compounds may be combined in a single preparation or contained in two separate formulations. Pharmaceutical compositions which comprise the active compound 1 and an active compound 2 in a single preparation are preferred.

Accordingly the present invention relates to a pharmaceutical composition comprising, together, an effective amount of the active compound 1 in combination with an effective amount of an active compound 2, and, optionally at least one pharmaceutically acceptable carrier.

The present invention also provides a device comprising a pharmaceutical formulation described before.

The present invention further provides a kit comprising active compound 1 and an active compound 2 in separate unit dosage forms, said forms being suitable for administration of the active compounds 1 and 2 in effective amounts.

The present invention also relates to the use of active compound 1 in combination with an active compound 2 as a medicament.

Moreover the present invention relates to the use of the active compound 1 in combination with an active compound 2 in the preparation of a pharmaceutical composition or a kit for the prophylaxis or treatment, by separate, simultaneous or sequential administration of the active compound 1 and an active compound 2, of an inflammatory or obstructive airways disease, such as asthma or chronic obstructive pulmonary disease (COPD).

Finally the present invention provides methods for the prevention and/or treatment of an inflammatory or obstructive airways disease treating a respiratory disorder such as asthma or chronic obstructive pulmonary disease (COPD), said method which comprising simultaneous or sequential administration of an effective amount of the active compound 1 in combination with a an active compound 2 to a subject in need thereof.

In particular the chloride salt of active compound 1 has been found to be equieffective to tiotropium bromide in terms of receptor potency and duration of action, but significantly short-acting on the M2 receptors. Therefore a salt of active compound 1 may provide significant therapeutic benefit in the treatment of respiratory diseases such as asthma and COPD, when administered by inhalation.

In particular, said active drug could provide antimuscarinic-based pharmaceutical compositions provided with a rapid onset of action and a long-lasting effect in such a way as it could be administered once a day with a great improvement of the compliance of patients. Moreover said pharmaceutical compositions could turn out to be safer with respect to those of the prior art.

It has now being found that an unexpectedly beneficial therapeutic effect, particularly a synergistic effect, is observed in the treatment of inflammatory or obstructive diseases of the respiratory tract if compound 1 is used in combination with a long-acting beta₂-agonist.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the existence of a synergic action for a preferred embodiment of the present invention.

### DETAILED DESCRIPTION OF THE PREFERRED EMBODIMENTS

In the context of the present invention, the term “long-acting beta₂-agonist” means a compound that is administered not more frequently than twice a day, preferably once a day.

The terms “active drug”, “active ingredient”, “active”, “active compound”, “active substance”, and “therapeutic agent” are synonyms and used interchangeably.

The terms “muscarinic receptor antagonists”, “antimuscarinic drugs” and ‘anticholinergic drugs’ are synonyms and used interchangeably.

By “daily therapeutically effective dose”, hereinafter called “daily dose”, it is meant the quantity of active ingredient administered daily by inhalation and delivered in one or more actuations, preferably one or two actuations (shots) of an inhaler.

By “actuation” it is meant the release of the active ingredient from the device by a single activation (e.g. mechanical or breath).

As used herein the term “substantially optically pure” means an active ingredient having an optical purity higher than 95% w/w, preferably higher than 98% w/w.

As used herein, the term “fixed combination” means a combination wherein the active substances are in a fixed amount and quantitative ratio.

As used herein the term “synergistic” means that the activity of the two compounds is more than would be expected by summing their respective individual activities in a given assay.

Thus in a first embodiment, the present invention provides compositions which comprise the active compound 1 in combination with an active compound 2. In particular the present invention provides compositions comprising a fixed combination of active compound 1 and an active compound 2.

Within the scope of the present invention, any reference to the active compound 1 is to be regarded as a reference to a pharmaceutically acceptable salt of of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane, whose formula is reported below:

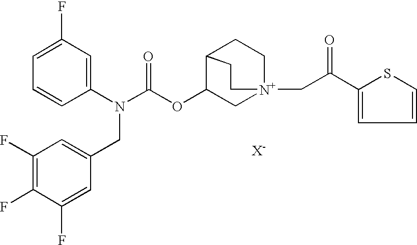

wherein X⁻ is a pharmaceutically acceptable anion, preferably selected from the group consisting of chloride, bromide, iodide, sulfate, phosphate, methanesulfonate, nitrate, maleate, acetate, citrate, fumarate, tartrate, oxalate, succinate, benzoate, and p-toluenesulfonate. The active compound 1 is preferably used in the form of its chloride salt.

It will be apparent to those skilled in the art that the active compound 1 displays an asymmetric carbon on the quinuclidine ring, and hence may be in the form of two optical stereoisomers, (3R)— and (3S)-stereoisomers or a racemic mixture thereof. In the preferred embodiments the active compound 1 is in the form of the substantially pure (3R)-enantiomer. The (3R)-enantiomer of active compound 1 in the form of chloride salt is hereinafter referred to as compound 1′.

Within the scope of the present invention, any reference to the active compound 2 is to be regarded as a reference to a long-acting phenylalkylamino beta₂-agonist or a pharmaceutically acceptable salt thereof, also including the relevant enantiomers or mixtures thereof.

Examples of physiologically acceptable acid addition salts of the active compound 2 according to the invention are the pharmaceutically acceptable salts which are selected among the salts of hydrochloric acid, hydrobromic acid, sulfuric acid, phosphoric acid, methanesulfonic acid, acetic acid, fumaric acid, succinic acid, lactic acid, citric acid, tartaric acid, or maleic acid.

The active compounds 2 may also be present according to the invention in the form of hydrates or solvates thereof.

The compositions according to the invention may comprise the compound 1 and the compound 2 in weight ratios ranging from 1:400 to 40:1.

Advantageously the compound 2 is selected from the group consisting of formoterol, hereinafter also indicated as compound 2a, and salmeterol, hereinafter also indicated as compound 2b, or salts thereof. The salts of salmeterol and formoterol, also include the relevant enantiomeric salts of (R)-salmeterol, (S)-salmeterol, (R,R)-formoterol, (S,S)-formoterol, (R,S)-formoterol, (S,R)-formoterol, and the mixtures thereof, while the enantiomeric salts of (R)-salmeterol and the racemic mixture (R,R)(S,S)-formoterol are of particular importance.

Formoterol is preferably used in the form fumarate salt, more preferably in the form of dihydrate fumarate salt, hereinafter also indicated as compound 2a′, while salmeterol is preferably used as xinafoate salt, hereinafter also indicated as compound 2b′.

The active compound 2 may also be a quinolinone derivative belonging to the formula A:

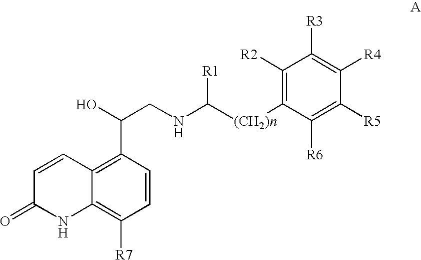

wherein:

R₁ is methyl and R₂ is hydrogen or R₁ and R₂ form a alkylene bridge, (CH₂)ₘ, with m equal to 1 or 2, preferably 1;

R₃, R₄, R₅, and R₆ are each independently hydrogen, hydroxy, a straight chain or branched C₁-C₄ alkyl, a straight chain or branched C₁-C₄ alkyl substituted with one or more halogen atoms and/or hydroxy groups, halogen, straight chain or branched C₁-C₄ alkoxy; and

R₇ is hydrogen, hydroxy, straight chain or branched C₁-C₄ alkyl, straight chain or branched C₁-C₄ alkoxy.

Particularly preferred is the active compound wherein R₁ is methyl, R₄ is methoxy, R₂, R₃, R₅, and R₆ are hydrogen, R₇ is hydroxy, and n=1, that is the 8-hydroxy-5-[1-hydroxy-2-[2-(4-methoxyphenyl)-1-methylethyl]amino]ethyl]-2(1H)-quinolinone, hereinafter are indicated as compound 2c.

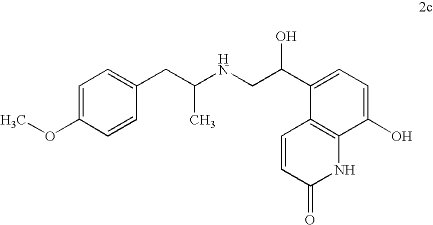

It will be apparent to those skilled in the art that said compound 2c displays two asymmetric carbons at the position —CH(CH₃)— and at the position —CH(OH)—, respectively. Therefore compound 2c may exist in the form of four different stereoisomers, e.g. (R,R)—, (R,S)—, (S,S)—, (S,R)—, or mixtures thereof. In a preferred embodiment the active compound 2c is in the form of the optical pure (R,R)-enantiomer which has been also reported with the name of carmoterol or the experimental codes of TA 2005 and CHF 4226. More preferably carmoterol is used as the hydrochloride salt, hereinafter indicated as compound 2c′.

Another preferred quinoline derivative is the compound wherein R₁ and R₂ form a methylenic bridge, R₃ and R₆ are H, R₄ and R₅ are ethyl, R₇ is OH, n=1 which has also been reported with the name of indacaterol. Indacaterol is hereinafter also indicated as compound 2d. Indacaterol is preferably used in the form of maleate salt, hereinafter also indicated as compound 2d′.

A further long-acting phenylalkylamino beta₂-agonist which might be advantageously used in combination with the active compound 1 is the compound milveterol (compound 2e) or a pharmaceutically accepable salt thereof. The salts of milveterol include the relevant enantiomeric salts (R,R), (S,S), (R,S), (S,R), and the mixtures thereof. Preferably milveterol is used as hydrochloride salt (compound 2e′).

Other long-acting phenylalkylamino beta₂-agonists which may be used in the combination of the invention are those known with the experimental codes PF-610355 and BI-1744-CL.

In a preferred embodiment of the present invention, the composition comprises compound 1′ in combination with formoterol fumarate dihydrate. In another preferred embodiment of the present invention, the composition comprises compound 1′ in combination with carmoterol hydrochloride.

Within the scope of the present invention, the active compounds 1 and 2 may be administered simultaneously or sequentially, while it is preferable according to the invention to administer active compounds 1 and 2 simultaneously.

In the composition of the present invention, the active compounds 1 and 2 may be combined in a single preparation or contained in two separate formulations. Pharmaceutical compositions which comprise the active compounds 1 and 2 in a single preparation, and optionally a pharmaceutically acceptable carrier, are preferred according to the invention.

The ratio by weight in which the active compounds 1 and 2 may be used in the pharmaceutical compositions according to the invention might be variable. The ratios may also vary on the basis of the different molecular weights of the various salt forms. The weight ratios specified below were based on compound 1′, the fumarate dihydrate salt of formoterol (compound 2a′), the xinafoate salt of salmeterol (compound 2b′), the hydrochloride salt of carmoterol (compound 2c′), the maleate salt of indacaterol (compound 2d′), and the hydrochloride salt of milveterol (compound 2e′).

For instance, the pharmaceutical compositions according to the present invention may comprise the compound 1′ and the compound 2a′ in weight ratios ranging from 1:30 to 7:1, preferably from 1:25 to 4:1, more preferably from 1:20 to 2:1.

In another embodiment, the compositions may comprise compound 1′ and the compound 2b′ in weight ratios ranging from 1:60 to 3:1, preferably from 1:50 to 1:1, more preferably from 1:40 to 1:2.

In another embodiment, the compositions may comprise compound 1′ and the compound 2c′ in weight ratios ranging from 1:10 to 40:1, preferably from 1:8 to 20:1, more preferably from 1:6 to 10:1.

In an further embodiment, the compositions may comprise the compound 1′ and the compound 2d′ in weight ratios ranging from 1:250 to 1:1, preferably from 1:200 to 2:5, more preferably from 1:150 to 1:5.

In a further embodiment, the compositions may comprise compound 1′ and the compound 2e′ in weight ratios ranging from 1:40 to 20:1, preferably from 1:20 to 10:1, more preferably from 1:10 to 5:1.

The compositions of the present invention may be administered one or more times a day, preferably once a day, and the daily dose of active compounds 1 and 2 may vary depending on the disease and the conditions (weight, sex, age) of the patient.

In one embodiment, the daily dose may be reached by a single or double administration.

In another preferred embodiment, the daily dose may be reached by a single administration and delivered in one actuation of the inhaler.

In another preferred embodiment, the daily dose may be reached by a single administration and delivered in more actuations of the inhaler, preferably two.

In another preferred embodiment, the daily dose may be reached by a double administration and delivered in one actuation of the inhaler.

In another preferred embodiment, the daily dose may be reached by a double administration and delivered in more actuations of the inhaler, preferably two.

The compositions according to the present invention are normally administered so that the delivered daily dose of active compound 1 is comprised between 1 μg and 20 μg, and that of active compound 2 is comprised between 0.5 μg and 400 μg.

Advantageously, when the compound 2 is formoterol, the compositions may deliver a daily dose of compound 1′, comprised between 1 μg and 20 μg, preferably between 1 μg and 10 μg, more preferably between 1 μg a 5 μg, and a daily dose of formoterol, calculated as compound 2a′, comprised between 3 μg and 24 μg. In one embodiment, the daily dose of compound 2a′ may be comprised between 3 μg and 6 μg. In another embodiment, the daily dose of compound 2a′ may be comprised between 6 μg and 12 μg. In certain further embodiments, the daily dose of compound 2a′ may be comprised between 12 μg and 18 μg or between 18 μg and 24 μg.

According to one embodiment, the compositions according to the invention may comprise a quantity of compound 1′ and compound 2a′ such that a daily dose comprised between 1 μg and 20 μg, preferably between 1 and 10 μg, more preferably between 1 and 5 μg of compound 1′ and of about 3 μg of compound 2a′ are delivered.

In another embodiment, a daily dose comprised between 1 μg and 20 μg, preferably between 1 and 10 μg, more preferably between 1 and 5 μg of compound 1′ and of about 4 μg of 2a′ are delivered.

In a further embodiment, a daily dose comprised between 1 μg and 20 μg, preferably between 1 and 10 μg, more preferably between 1 and 5 μg of compound 1′ and of about 6 μg of 2a′ are delivered.

In certain embodiments, a daily dose comprised between 2 μg and 5 μg of compound μ and of about 6 μg, or 8 μg, or 10 μg, or 12 μg, or 15 μg, or 18 μg or 24 μg of 2a′ are delivered.

Advantageously, when the compound 2 is salmeterol, the compositions may deliver a daily dose of compound 1′, comprised between 1 μg and 20 μg, preferably between 1 μg and 10 μg, more preferably between 1 and 5 μg, and a daily dose of salmeterol, calculated as compound 2b′, comprised between 12 μg and 50 μg. In one embodiment, the daily dose of compound 2b′ may be comprised between 12 μg and 25 μg. In another embodiment the daily dose of compound 2b′ may be comprised between 25 μg and 50 μg.

In one embodiment, the compositions according to the invention may contain a quantity of compound 1′ and compound 2b′ such that a daily dose comprised between 2 μg and 5 μg of compound 1′ and of about 12 μg of compound 2b′ are delivered.

In another embodiment, a daily dose comprised between 2 μg and 5 μg of compound 1′ and of about 25 μg of 2b′ are delivered.

In a further embodiment, a daily dose comprised between 2 μg and 5 μg of compound 1′ and of about 50 μg of 2b′ are delivered.

Advantageously, when the compound 2 is carmoterol the compositions may deliver a daily dose of compound 1′, comprised between 1 μg and 20 μg, preferably between 1 μg and 10 μg, more preferably between 1 and 5 μg, and a daily dose of carmoterol, calculated as compound 2c′, comprised between 0.5 μg and 8 μg.

In one embodiment, the daily dose of compound 2c′ is comprised between 0.5 μg and 2 μg. In another embodiment the daily dose of compound 2c′ may be comprised between 2 μg and 4 μg. In a further embodiment, the daily dose of compound 2c′ may be comprised between 4 μg and 8 μg.

In certain embodiments, daily doses of about 1 μg or 2 μg or 4 μg of compound 2c′ might be delivered. For example, without restricting the scope of the invention thereto, in one embodiment, the composition according to the invention may comprise a quantity of compound 1′ and compound 2c′ such that a daily dose of compound 1′ comprised between 2 μg and 5 μg and a daily dose of 2c′ comprised between 1 and 4 μg are delivered.

In a particular embodiment, a daily dose of about 2 μg of compound 1′ and of about 2 μg of 2c′ are delivered. In a further particular embodiment, a daily dose of about 2 μg or 5 μg of compound 1′ and of about 4 μg of 2c′ are delivered.

Advantageously, when the compound 2 is indacaterol, the compositions may deliver a daily dose of compound 1′, comprised between 1 μg and 20 μg, preferably between 1 μg and 10 μg, more preferably between 1 and 5 μg, and a daily dose of indacaterol, calculated as compound 2d′, comprised between 25 μg and 200 μg.

In one embodiment, the daily dose of compound 2d′ is comprised between 25 μg and 50 μg. In another embodiment the daily dose of compound 2d′ is comprised between 50 μg and 100 μg. In a further embodiment, the daily dose of compound 2d′ is comprised between 100 μg and 200 μg.

In one embodiment, the compositions according to the invention may comprise a quantity of compound 1′ and compound 2d′ such that a daily dose comprised between 2 μg and 5 μg of compound 1′ and of about 25 μg of 2d′ are delivered.

In another embodiment, a daily dose comprised between 2 μg and 5 μg of compound 1′ and of about 50 μg of 2d′ are delivered. In a further embodiment, a daily dose comprised between 2 μg and 5 μg of compound 1′ and of about 100 μg or 200 μg of 2d′ are delivered.

Advantageously, when the compound 2 is milveterol, the compositions may deliver a daily dose of compound 1′ comprised between 1 μg and 20 μg, preferably between 1 μg and 10 μg, more preferably between 1 and 5 μg, and a daily dose of milveterol, calculated as compound 2e′, comprised between 1 μg and 20 μg.

The quantities of active substance in the pharmaceutical compositions according to the invention which are administered per single dose can be calculated analogously if instead of the mentioned salts of the compounds 2, other salts are used.

The pharmaceutical compositions according to the present invention may optionally comprise a further active ingredient useful for the prevention and/or treatment of an inflammatory or obstructive airway disease.

According to a particular embodiment, the compositions of the present invention may further comprise a corticosteroid, preferably selected from the group consisting of beclomethasone dipropionate (BDP), budesonide and epimers thereof, flunisolide, fluticasone propionate, mometasone furoate, ciclesonide, triamcinolone acetonide, rofleponide palmitate. More preferably, the corticosteroid is beclomethasone dipropionate or budesonide.

The compositions of the present invention may be preferably administered by inhalation. Inhalable preparations include inhalable powders, propellant-containing metering aerosols or propellant-free inhalable formulations.

For administration as a dry powder, single- or multi-dose inhaler device known from the prior art may be utilized, wherein the powder can be filled in gelatine, plastic or other capsules, cartridges or blister packs or in a reservoir. Said devices are known as dry powder inhalers (DPIs).

A diluent or carrier, generally non-toxic and chemically inert to the medicaments, e.g. lactose or any other additive suitable for improving the respirable fraction can be added to the powdered medicament.

Inhalation aerosols containing a propellant gas such as hydrofluoroalkanes may contain the active ingredients of the combination of the present invention either in solution or in dispersed form. Said propellant-driven formulations may also contain other ingredients such as co-solvents, stabilizers such as inorganic acids, and optionally other excipients. They are usually administered by inhaler devices known as metered dose inhalers (MDIs).

The propellant-free inhalable formulations comprising the combination of the present invention may be in form of solutions or suspensions in an aqueous, alcoholic or hydroalcoholic medium and they may be delivered by jet or ultrasonic nebulizers known from the prior art or by inhaler devices known as soft-mist nebulizers (for example the nebuliser sold under the registered name of Respimat™).

Treatment of inflammatory or obstructive airways diseases in accordance with the present invention may be symptomatic or prophylactic. Inflammatory or obstructive airways diseases to which the claimed combinations are applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e. g. of less than 4 or 5 years of age, exhibiting wheezing symptoms and diagnosed or diagnosable as “wheezy infants”, an established patient category of major medical concern. Prophylactic efficacy in the treatment of asthma will be evidenced by reduced frequency or severity of symptomatic attack, e. g. of acute asthmatic or bronchoconstrictor attack, improvement in lung function or improved airways hyperreactivity. It may further be evidenced by reduced requirement for other, symptomatic therapy. Prophylactic benefit in asthma may in particular be apparent in subjects prone to “morning dipping”. “Morning dipping” is a recognized asthmatic syndrome, common to a substantial percentage of asthmatics and characterized by asthma attack, e. g., between the hours of about 4 to 6 a.m., i. e. at a time normally substantially distant form any previously administered symptomatic asthma therapy.

Other inflammatory or obstructive airways diseases and conditions to which the present invention is applicable include acute lung injury (ALI), adult respiratory distress syndrome (ARDS), chronic obstructive pulmonary, airways or lung disease (COPD, COAD or COLD) including chronic bronchitis and emphysema, bronchiolitis, bronchiectasis and exacerbation of airways hyperreactivity consequent to other drug therapy, in particular other inhaled drug therapy.

Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tobacosis and byssinosis.

In particular, the combination of the present invention is useful for the prevention and/or treatment of the chronic obstructive pulmonary disease (COPD), including chronic bronchitis and emphysema, bronchiolitis, and bronchiectasis, as in COPD patients, the antimuscarinic drugs relieve the airways constriction due to the vagal cholinergic tone.

The invention further provides a pharmaceutical kit comprising the active compound 1 and an active compound 2 in separate unit dosage forms, said forms being suitable for separate, simultaneous or sequential administration of the active compounds 1 and 2 in effective amounts.

Such a kit suitably further comprises one or more inhalation devices for administration of active compounds 1 and 2. For example, the kit may comprise one or more dry powder inhalation devices adapted to deliver dry powder from a capsule, together with capsules containing a dry powder comprising a dosage unit of the active compound 1 and capsules containing a dry powder comprising a dosage unit of an active compound 2.

In another example, the kit may comprise a multidose dry powder inhalation device containing in the reservoir thereof a dry powder comprising the active compound 1 and a multidose dry powder inhalation device containing in the reservoir thereof a dry powder comprising an active compound 2.

In a further example, the kit may comprise a metered dose inhaler containing an aerosol comprising the active compound 1 in a propellant, and a metered dose inhaler containing an aerosol comprising an active compound 2.

Other features of the invention will become apparent in the course of the following descriptions of exemplary embodiments which are given for illustration of the invention and are not intended to be limiting thereof.

### EXAMPLES

### Example 1

### Formulations for Metered Dose Inhalers

Different formulations for metered dose inhalers are prepared with the following compositions:

| | Amounts | | |
|---|---|---|---|
| | Per unit | | Daily dose |
| Formulation (a) | mg | % (w/w) | μg |
| Compound 1′ | 1.60 | 0.017 | 10 |
| Formoterol fumarate•2H₂O | 0.96 | 0.01 | 6 |
| Ethanol | 1113.6 | 12 | |
| Hydrochloric acid 1 M | 0.014 | 0.00015 | |
| HFA 134a | q.s. to 9280 | | |

| | Amounts | | |
|---|---|---|---|
| | Per unit | | Daily dose |
| Formulation (b) | mg | % (w/w) | μg |
| Compound 1′ | 0.31 | 0.0028 | 2 |
| Carmeterol hydrochloride | 0.31 | 0.0028 | 2 |
| (compound 2c′) | | | |
| Ethanol | 1650.0 | 15 | — |
| Phosphoric acid 15.2 M | 0.3 | 0.0027 | — |
| HFA 134a | q.s. to 11,000 | | |

| | Amounts | | |
|---|---|---|---|
| | Per unit | | Daily dose |
| Formulation (c) | mg | % (w/w) | μg |
| Compound 1′ | 1.54 | 0.016 | 10 |
| Carmoterol hydrochloride | 0.70 | 0.006 | 4 |
| (compound 2c′) | | | |
| Ethanol | 1650.0 | 15 | |
| Phosphoric acid 15.2 M | 0.3 | 0.0027 | |
| HFA 134a | q.s. to 11,000 | 84.981 | |

| | Amounts | | |
|---|---|---|---|
| | Per unit | | Daily dose |
| Formulation (d) | mg | % (w/w) | μg |
| Compound 1′ | 6.17 | 0.064 | 40 |
| Carmoterol hydrochloride | 0.70 | 0.006 | 4 |
| (compound 2c′) | | | |
| Ethanol | 1650.0 | 15 | |
| Phosphoric acid 15.2 M | 0.3 | 0.0027 | |
| HFA 134a q.s. to 9.72 ml | q.s. to 11,000 | 84.933 | — |

### Example 2

### Powder Formulations for a Dry Powder Inhalers

Different formulations for dry powder inhalers are prepared with the following compositions:

Amounts

| | |
|---|---|
| For shot of the | |
| inhaler | Daily dose |

| Formulation (a) | mg | % (w/w) | μg |
|---|---|---|---|
| Compound 1′ | 0.01 | 0.1 | 10 |
| Formoterol fumarate•2H₂O | 0.006 | 0.06 | 6 |
| (compound 2a′) | | | |
| Alpha-lactose monohydrate | 9.959 | 99.59 | |
| Magnesium stearate | 0.025 | 0.25 | |
| Total weight | 10 | 100 | |

Amounts

| | |
|---|---|
| For shot of | |
| the inhaler | Daily dose |

| Formulation (b) | mg | % (w/w) | μg |
|---|---|---|---|
| Compound 1′ | 0.01 | 0.1 | 10 |
| Carmoterol hydrochloride | 0.004 | 0.04 | 4 |
| (compound 2c′) | | | |
| Alpha-lactose monohydrate | 9.961 | 99.61 | |
| Magnesium stearate | 0.025 | 0.25 | |
| Total weight | 10 | | |

### Example 3

### Assessment of the Bronchodilation Activity of Compound 1′

Airway reactivity is measured using barometric plethysmography (Buxco, USA). Male guinea pigs (500-600 g) are individually placed in plexiglass chambers. After an acclimatisation period, animals are exposed to nebulised saline for 1 minute to obtain airway baseline reading. This is followed by a 1 minute challenge with nebulised acetylcholine (Ach)-2.5 mg/mL.

After 60 minutes, 5 minutes nebulisation of vehicle or the compound 1′ in the range 2.5-250 μM are applied and Ach challenge is then repeated after 2, 5, 24, 48, and 72 hours (h). Recording of pressure fluctuations in the chambers are taken for 5 minutes after each nebulisation and analysed to calculate Enhanced Pause (Penh). Airway reactivity is expressed as percentage increase in Penh compared with Penh values from the nebulisation of vehicle.

Two hours after the end of nebulisation with compound 1′, the Ach-induced increase in Penh is dose-dependently inhibited by the compound, with a maximal effect of 99.6±0.4 at 50 μM. As for the time-course of the effect, compound 1′ shows increasing duration of action with increasing dose.

After inhalation of 250 μM of compound 1′, effect persists unchanged up to 48 hours (83.0±16.1%), while at 72 hours a residual activity of 34.8±20.9% is present. Twenty-four hours after 25 and 50 μM compound 1′ inhalation, a significant bronchoprotective effect was observed (63.7±15.1% and 87.1±8.7%, respectively). At 50 μM, a significant inhibition persists up to 48 hours (49.2±23.2%). Inhalation of lower concentrations results in an effect that did not exceed the 5 hours observation point. The estimation of lung levels of compound 1′ achieved after nebulisation endowed with a submaximal bronchodilator activity at 2 hours after treatment reveals that the its retained dose in the target organ is about 50 μg/kg. If an extrapolation of these results from guinea-pig to human is made, it can be predicted that in patients the daily dose might be comprised between 1 and 20 μg, preferably between 1 and 10 μg and more preferably between 1 and 5 μg.

### Example 4

### Synergistic Activity of Fixed Dose Combination of Carmoterol/Compound 1′ on Carbachol-Induced Contraction in Guinea-Pigs Trachea

Male guinea-pigs (380-420 g) are used. The investigation conforms to the Guide for the Care and Use of Laboratory Animals published by the US National Institutes of Health (NIH Publication No. 85-23, revised 1996).

The trachea is removed, cleaned and cut into two zig-zag strips, according to Emmerson et al. (J. Pharm. Pharmacol., 1979; 31:798). A sub-maximal concentration of carbachol (3×10⁻⁷ M) is then added to the organ bath and when stable contraction is reached, a cumulative concentration-response curve (from 1×10⁻¹¹ to 1×10⁻⁷ M) to carmoterol or compound 1′ is generated (n=12 for each compound). Maximal relaxation is tested by adding a maximal dose of isoproterenol (3×10⁻⁷ M) at the end of the curve. Data are expressed as percentages of the isoproterenol-induced maximal relaxation. Isobolographic analysis is used to characterize the pharmacological interaction between carmoterol and compound 1′.

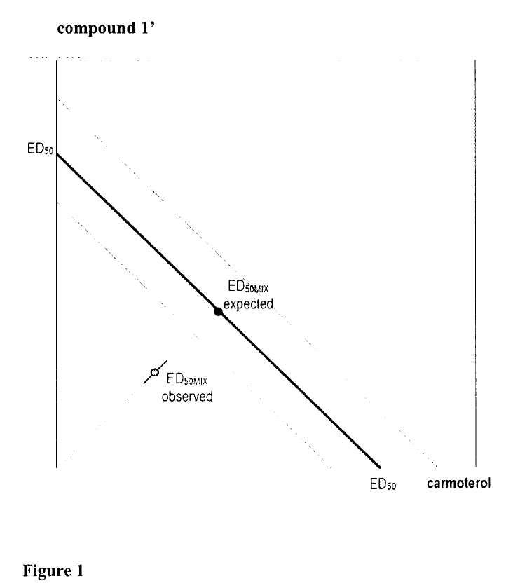

To estimate the experimental ED₅₀MIX, the dose-effect curve resulting from this second set of experiments (Table 1) is fitted with a mixed logistic model. All computations are carried out resorting to NLMIXED procedure (SAS/STAT® User's Guide, version 9, 2004. SAS Institute Inc., Cary, N.C.). The ED₅₀ (95% conf. lim.) for carmoterol is 5.4×10⁻¹⁰ M (4.6×10⁻¹⁰ to 6.4×10⁻¹⁰ M), whereas the ED₅₀ (95% conf. lim.) for compound 1′ is 9.3×10⁻¹⁰ M (7.9×10⁻¹⁰ to 10.9×10⁻¹⁰ M). Isobolographic analysis (FIG. 1) shows the existence of a synergic action between compound 1′ and carmoterol, the ED₅₀MIX observed being significantly lower than the value expected under hypothesis of additivity.

This study shows that the β2-selective agonist carmoterol and the M3-selective antagonist compound 1′ are both potent in antagonizing carbachol-induced contraction in guinea-pig airways. Moreover, in line with their complementary molecular mechanism of action, in the frame of a functional agonism-antagonism, fixed combinations display synergistic effect in the control of cholinergic contraction in guinea-pig trachealis.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A composition comprising:
(a) a pharmaceutically acceptable salt of formula 1: 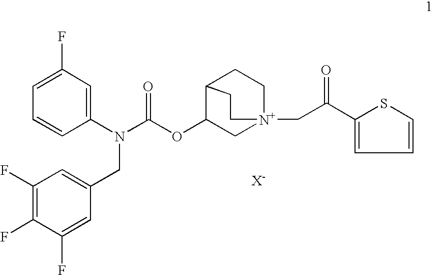
wherein:
X⁻ is a pharmaceutically acceptable anion; and
(b) a long-acting phenylalkylamino beta₂-agonist.

2. The composition according to claim 1, wherein said anion is selected from the group consisting of chloride, bromide, iodide, sulfate, phosphate, methanesulfonate, nitrate, maleate, acetate, citrate, fumarate, tartrate, oxalate, succinate, benzoate, and p-toluenesulfonate.

3. The composition according to claim 1, wherein said long-acting phenylalkylamino beta₂-agonist is a salt selected from the group consisting of a hydrochloric acid salt, a hydrobromic acid salt, a sulfuric acid salt, a phosphoric acid salt, a methanesulfonic acid salt, an acetic acid salt, a fumaric acid salt, a succinic acid salt, a lactic acid salt, a citric acid salt, a tartaric acid salt, and a maleic acid salt.

4. The composition according to claim 1, wherein said pharmaceutically acceptable salt of formula 1 and said long-acting phenylalkylamino beta₂-agonist are present in a fixed combination.

5. The composition according to claim 4 wherein said pharmaceutically acceptable salt of formula 1 and said long-acting phenylalkylamino beta₂-agonist are present in a weight ratio of 1:400 to 40:1.

6. The composition according to claim 1, which comprises the (3R)-enantiomer of said pharmaceutically acceptable salt of formula 1 in the form of chloride salt.

7. The composition according to claim 6, which comprises said pharmaceutically acceptable salt of formula 1 in an amount suitable for administration of said pharmaceutically acceptable salt of formula 1 at a daily dose of 1 μg to 20 μg.

8. The composition according to claim 7, which comprises said pharmaceutically acceptable salt of formula 1 in an amount suitable for administration of said pharmaceutically acceptable salt of formula 1 at a full daily dose of 1 μg to 10 μg.

9. The composition according to claim 8, which comprises said pharmaceutically acceptable salt of formula 1 in an amount suitable for administration of said pharmaceutically acceptable salt of formula 1 at a daily dose of 1 μg to 5 μg.

10. The composition according to claim 1, which comprises said long-acting phenylalkylamino beta₂-agonist in an amount suitable for administration of said long-acting phenylalkylamino beta₂-agonist in a daily dose of 0.5 μg to 400 μg.

11. The composition according to claim 4, which comprises formoterol.

12. The composition according to claim 4, which comprises formoterol fumarate dihydrate.

13. The composition according to claim 12, wherein the weight ratio between said pharmaceutically acceptable salt of formula 1 and said formoterol fumarate dihydrate ranges from 1:30 to 7:1.

14. The composition according to claim 12, wherein the weight ratio between said pharmaceutically acceptable salt of formula 1 and said formoterol fumarate dihydrate ranges from 1:25 to 4:1.

15. The composition according to claim 12, wherein the weight ratio between said pharmaceutically acceptable salt of formula 1 and said formoterol fumarate dihydrate ranges from 1:20 to 2:1.

16. The composition according to claim 4, which comprises salmeterol.

17. The composition according to claim 4, which comprises salmeterol in the form of xinafoate salt.

18. The composition according to claim 17, wherein the weight ratio between said pharmaceutically acceptable salt of formula 1 and said salmeterol xinafoate salt ranges from 1:60 to 3:1.

19. The composition according to claim 17, wherein the weight ratio between said pharmaceutically acceptable salt of formula 1 and said salmeterol xinafoate salt ranges from 1:50 to 1:1.

20. The composition according to claim 17, wherein the weight ratio between said pharmaceutically acceptable salt of formula 1 and said salmeterol xinafoate salt ranges from 1:40 to 1:2.

21. The composition according to claim 4, which comprises a compound of formula A: 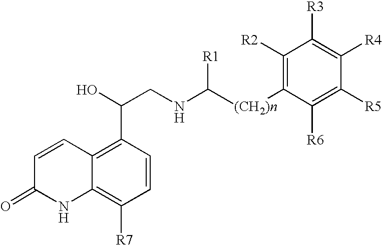
wherein
R₁ is methyl and R₂ is hydrogen or R₁ and R₂ form a alkylene bridge, —(CH₂)ₘ— where m is 1 or 2;
R₃, R₄, R₅, and R₆ are each independently hydrogen, hydroxy, a straight chain or branched C₁-C₄ alkyl, a straight chain or branched C₁-C₄ alkyl substituted with one or more halogen atoms and/or hydroxy groups, halogen, straight chain or branched C₁-C₄ alkoxy; and
R₇ is hydrogen, hydroxy, straight chain or branched C₁-C₄ alkyl, straight chain or branched C₁-C₄ alkoxy.

22. The composition according to claim 21, which comprises a compound of formula A wherein R₁ is methyl, R₄ is methoxy, R₂, R₃, R₅, R₆ are hydrogen, R₇ is hydroxy and n=1.

23. The composition according to claim 22, which comprises a compound of formula A wherein R₁ is methyl, R₄ is methoxy, R₂, R₃, R₅, R₆ are hydrogen, R₇ is hydroxy and n=1 in the form of a hydrochloride salt.

24. The composition according to claim 23, wherein the weight ratio between said pharmaceutically acceptable salt of formula 1 and said compound of formula A wherein R₁ is methyl, R₄ is methoxy, R₂, R₃, R₅, R₆ are hydrogen, R₇ is hydroxyl, and n=1 in the form of a hydrochloride salt ranges from 1:10 to 40:1.

25. The composition according to claim 23, wherein the weight ratio between said pharmaceutically acceptable salt of formula 1 and said compound of formula A wherein R₁ is methyl, R₄ is methoxy, R₂, R₃, R₅, R₆ are hydrogen, R₇ is hydroxyl, and n=1 in the form of a hydrochloride salt ranges from 1:8 to 20:1.

26. The composition according to claim 23, wherein the weight ratio between said pharmaceutically acceptable salt of formula 1 and said compound of formula A wherein R₁ is methyl, R₄ is methoxy, R₂, R₃, R₅, R₆ are hydrogen, R₇ is hydroxyl, and n=1 in the form of a hydrochloride salt ranges from 1:6 to 10:1.

27. The composition according to claim 21, which comprises a compound of formula A wherein R₁ and R₂ form a methylenic bridge, R₃ and R₆ are H, R₄ and R₅ are ethyl, R₇ is OH, and n=1.

28. The composition according to claim 27, which comprises a compound of formula A wherein R₁ and R₂ form a methylenic bridge, R₃ and R₆ are H, R₄ and R₅ are ethyl, R₇ is OH, and n=1 in the form of a maleate salt.

29. The composition according to claim 28, wherein the weight ratio between said pharmaceutically acceptable salt of formula 1 and said compound of formula A wherein R₁ and R₂ form a methylenic bridge, R₃ and R₆ are H, R₄ and R₅ are ethyl, R₇ is OH, and n=1 in the form of a maleate salt ranges from 1:250 to 1:1.

30. The composition according to claim 28, wherein the weight ratio between said pharmaceutically acceptable salt of formula 1 and said compound of formula A wherein R₁ and R₂ form a methylenic bridge, R₃ and R₆ are H, R₄ and R₅ are ethyl, R₇ is OH, and n=1 in the form of a maleate salt ranges from 1:200 to 2:5.

31. The composition according to claim 28, wherein the weight ratio between said pharmaceutically acceptable salt of formula 1 and said compound of formula A wherein R₁ and R₂ form a methylenic bridge, R₃ and R₆ are H, R₄ and R₅ are ethyl, R₇ is OH, and n=1 in the form of a maleate salt ranges from 1:150 to 1:5.

32. The composition according to claim 4, which comprises milveterol or a salt thereof.

33. A pharmaceutical composition comprising in admixture in a single preparation:
(a) a pharmaceutically acceptable salt of formula 1: 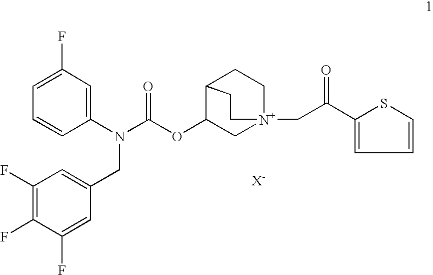
wherein:
X⁻ is a pharmaceutically acceptable anion; and
(b) a long-acting phenylalkylamino beta₂-agonist; and
(c) a pharmaceutically acceptable carrier.

34. The pharmaceutical composition according to claim 33, which comprises the (3R)-enantiomer of said pharmaceutically acceptable salt of formula 1 in the form of chloride salt.

35. The pharmaceutical composition according to claim 33, wherein said anion is selected from the group consisting of chloride, bromide, iodide, sulfate, phosphate, methanesulfonate, nitrate, maleate, acetate, citrate, fumarate, tartrate, oxalate, succinate, benzoate, and p-toluenesulfonate.

36. The pharmaceutical composition according to claim 33, further comprising a corticosteroid.

37. The pharmaceutical composition according to claim 36, which comprises a corticosteroid selected form the group consisting of beclomethasone dipropionate, budesonide and epimers thereof, flunisolide, fluticasone propionate, mometasone furoate, ciclesonide, triamcinolone acetonide, and rofleponide palmitate.

38. The pharmaceutical composition according to claim 33, wherein the pharmaceutical composition is an inhalable aerosol formulation comprising a propellant.

39. The pharmaceutical composition according to claim 33, wherein the pharmaceutical composition is an inhalable powder.

40. The pharmaceutical composition according to claim 33, wherein the pharmaceutical composition is an inhalable propellant-free solution or suspension.

41. A device comprising a pharmaceutical composition according to claim 33.

42. A kit comprising:
(a) a therapeutically effective amount of a pharmaceutically acceptable salt of formula 1 in a first unit dosage form: 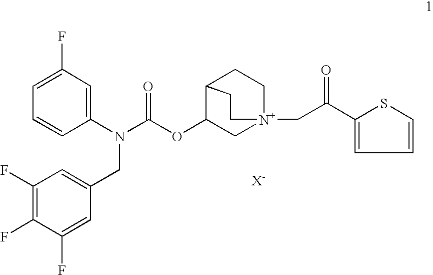
wherein:
X⁻ is an anion selected from the group consisting of chloride, bromide, iodide, sulfate, phosphate, methanesulfonate, nitrate, maleate, acetate, citrate, fumarate, tartrate, oxalate, succinate, benzoate, and p-toluenesulfonate; and
(b) a therapeutically effective amount of a long-acting phenylalkylamino beta₂-agonist in a second unit dosage form.

43. The kit according to claim 42, further comprising one or more inhaler devices.

44. A method for the prophylaxis or treatment of an inflammatory or obstructive airways disease, comprising simultaneous or sequential administration of
(a) an effective amount of a pharmaceutically acceptable salt of formula 1: 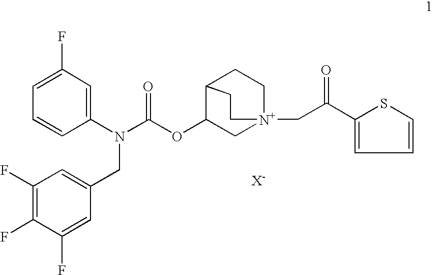
wherein:
X⁻ is a pharmaceutically acceptable anion; and
(b) an effective amount of a long-acting phenylalkylamino beta₂-agonist, to a subject in need thereof.

45. The method according to claim 44, wherein said disease is asthma.

46. The method according to claim 44, wherein said disease is chronic obstructive pulmonary disease (COPD).

47. The method according to claim 44, wherein said pharmaceutically acceptable salt of formula 1 is administered in a daily dose of 1 μg to 20 μg.

48. The method according to claim 44, wherein said pharmaceutically acceptable salt of formula 1 is administered in a daily dose of 1 μg and 10 μg.

49. The use according to claim 44, wherein said pharmaceutically acceptable salt of formula 1 is administered in a daily dose of 1 μg and 5 μg.
